(19) United States
(12) Patent Application Publication
(10) Pub. No.: US 2026/0363992 A1
(43) Pub. Date: Nov. 19, 2026

(54) Healthcare Formulations and Methods of Using the Same

(71) Applicant: Tri-K Industries, Denville, NJ (US)

(72) Inventors: Geera RAMAKRISHNAN, Denville, NJ (US); Andrea Serrano TRUJILLO, Wharton, NJ (US); Ashley BLOUIN, Manchester, NH (US); Lauren MURPHY, Auburn, NH (US); Saso EFREMOVSKI, Clifton, NJ (US)

(21) Appl. No.: 19/721,906

(22) Filed: Jun. 26, 2026

Related U.S. Application Data
(63) Continuation of application No. PCT/US2024/060814, filed on Dec. 18, 2024.
(60) Provisional application No. 63/615,946, filed on Dec. 29, 2023.

(51) Int. Cl. A61K 8/66; A61Q 5/00; A61Q 5/02; A61Q 5/12

(52) U.S. Cl. CPC A61K 8/66; A61Q 5/002; A61Q 5/004; A61Q 5/02; A61Q 5/12

(57) ABSTRACT

Presented herein are processes for forming active agent extracts and methods of using the same in healthcare formulations, such as hair care formulations. Active agent extracts are incorporated into compositions or formulations which, on application to damaged hair, reverses oxidative damage of disulfide bonds. More specifically, presented herein are processes for preparation of compositions of enzymatically hydrolyzed moringa seed extracts, which 1) repair cysteic acid residues and restore disulfide bonds, enhancing hair protein structural integrity, 2) protect hair from harsh hair treatments such as bleaching and other oxidative damage causing treatments such as coloring, highlighting, straightening, perming, hot ironing, and styling, and 3) actively penetrate hair, leading to improvement or restoration of internal hair structures and in mechanical properties of the hair.

### CROSS REFERENCE TO RELATED APPLICATION

This application is claims the benefit of U.S. Provisional Application No. 63/615,946 filed Dec. 29, 2023, the entire contents of which are incorporated herein by reference.

### FIELD OF THE INVENTION

The present invention relates to process of extraction and preparation of active agents and method methods of using the same for healthcare preparations.

### BACKGROUND OF THE DISCLOSURE

Hair is composed largely of proteins (65%-95%) and contains three major structural components: the cortex, medulla, and cuticle. The cortex provides the bulk of the hair mass and gives hair its tensile strength. The damage to the hair proteins in the cortex has been identified as the major concern in the hair care industry. A large number of hair treatments, as well as everyday aggressors contribute to hair protein damage.

Chemical treatments, heat and sunlight exposure cause permanent changes to covalent bonds in hair, leading to structural deviation and damage to hair proteins. Ultraviolet (UV) exposure, coloring and bleaching will oxidize disulfide bonds and generate cysteic acid residues; a change that has been considered irreversible in the hair care industry. The oxidative conversion of disulfide bonds to cysteic acid residues causes protein conformational distortion, resulting in weakening of the protein stability and deterioration of overall hair health. Hair becomes frizzy, dull, prone to breakage, easily tanged, and loses soft texture.

A wide range of approaches have been introduced to address these negative physical attributes of hair damage issues. Some of the approaches include hair conditioning via oily ingredients and hair protein modification via chemical ingredients. The hair conditioning products introduce oily texture to hair, masking or hiding the negative texture of damaged hair. The chemical ingredients promote ionic, covalent, hydrophobic and hydrogen bonds to mend or strengthen the hair protein structure.

The approaches above do not address or repair the hair protein damage. Instead, they merely cover up the negative attributes without addressing the basis of the hair damage. Ionic bond forming and hydrogen bond forming products become readily disabled under aqueous conditions such as hair washing. Hydrophobic interaction and covalent bond forming products in the market cause further protein distortion. They chemically and permanently alter free cysteine residues, inserting their chemical linkers in between hair proteins. Such a modification causes hair proteins to be further separated, leading to bulging, loss of stable alpha-helix protein secondary structure, and eventual disruption of macro-filament structure of protein network within hair cortex. U.S. Patent Application Publication No. 2023/0070929A1 discusses an extract of Moringa peregrina seed cake predominantly comprising mainly a peptide hydrolysate a process for obtaining the extract. However, the process discussed in this reference utilized a chemical proteolysis step during extractions. Further, German publication DE 102011089366A1 discusses the use of composition containing an active agent from Moringa oleifera for treating hair. However, specific reversal of oxidative damage is not demonstrated in the reference.

There is a need for the process for effective extraction of hair care active ingredients. There is also a need for hair treatment that reverses the oxidative damage and repairs the hair proteins without distorting the protein structure. Further, there is a need for formulations or compositions that can protect hair from UV damage and chemical hair treatment damage.

### SUMMARY OF THE DISCLOSURE

One of the objectives of the disclosure is to provide a process for effective extraction sequential extraction of active agents and the use of such active agents extracts in healthcare compositions or formulations, such as hair care compositions or formulations. Another objective of the disclosure is to provide methods of using active agents extracts according to the disclosure to reverse oxidative damage of permanently damaged hairs. Another objective of the disclosure is to reverse cysteine oxidative damage of hair. Another objective of the disclosure is to provide range of formulations containing active agents extracts. The objectives above are achieved with hair care formulations according to various aspects of the disclosure and methods of using the same.

Various aspects of the disclosure are directed to processes of extracting of active agents and using the active agents extracts as is or in hair care formulations for treating oxidatively damaged hair. On application to damaged hair, active agents extracts according to the disclosure have been found to reverse the oxidative damage such as in hair proteins or in skin proteins and provide protein repair at molecular level, leading to healthier hair with increased tensile strength, less breakage, improved appearance, and smooth feel without leaving the hair feeling greasy. Active agents extracts according to the disclosure have also been found to provide hair with thermal protection and long lasting moisturization. Relatedly, it has been found that hair care formulations, comprising active agents extracts according to various aspects of the disclosure, contain one or more compounds that penetrate into hair cortex, induce reversal of the cysteine oxidative damages, namely cysteic acid residues, promote disulfide bond formation, improve the protein stability, strengthens the hair fiber, and protect the hair from further oxidative damage.

Active agents extracts described herein can be used as-is or incorporated or formulated into a various healthcare composition or formulations accordingly. For example, active agents extracts described herein can be used as-is or as a component of multicomponent compositions or formulations for use in various healthcare applications such as hair care, skin care, body care applications. Compositions and formulations comprising active agents extracts described herein can take various forms including, but not limited to, emulsions, gels, sprays, bars, sticks, balms, creams, lotions, serums and solutions.

Various non-limiting inventive aspects according to the disclosure are as follows.

In some instances, a first aspect of the disclosure can be described as a process for preparing an extract of active agents for use in a healthcare product, the process comprising:
a) mixing water and moringa seed powder at about 40 °C to about 60 °C to form a solution, where the solution comprises about 75 wt % to about 87 wt % of water and about 13 wt % to about 25 wt % of moringa seed powder;
b) adjusting the pH of the solution of step a) to a pH ranging from about 2.5 to about 3.5 using an acid solution;
c) adding about 1.5 wt % to about 4 wt % of a fungal acid protease, based on the weight of the moringa seed powder, to the product of step b) and hydrolyzing it for about 2 to about 4 hours at about 40 °C to about 60 °C;
d) adjusting the pH of the product of step c) to a pH ranging from about 3.0 to about 6.0 using a base solution;
e) adding about 0.5 wt % to about 3 wt % of a cellulase enzyme, based on weight of the moringa seed powder, to the product of step d) and hydrolyzing it for about 1 to about 2 hours at about 60 °C to about 67 °C;
f) adjusting the pH of the product of step e) to about 8.0 to about 10 using a base solution at about 55 °C to about 65 °C;
g) adding about 3.5 wt % to about 6.5 wt % of an alkaline protease, based on weight of the moringa seed powder, to the product of step f) and hydrolyzing it for about 2 to about 6 hours at about 55 °C to about 65 °C;
h) adjusting the pH of the product of step g) to a pH<7.0 using an acid solution;
i) heating the product of step h) to about 80 °C to about 90 °C for about 15 to about 20 minutes;
j) cooling the product of step i) to a temperature of <50 °C and adjusting the pH thereof to a pH ranging from about 4.0 to about 5.0;
k) adding 0.5 wt % to 1.5 wt % of preservative, based on the total weight of the solution, to the product of step j);
l) adjusting the pH of product of step k) to 3.5 to 4.0 using an acid solution;
m) ageing product of step 1) for 0 to 50 days; and
n) filtering the product of step m) to obtain an active agents extract.

In some instances, a second aspect of the disclosure can be described as a process according to the first aspect, wherein the fungal acid protease is Aspergillus niger fungal acid protease; and/or the cellulase enzyme is Trichoderma longibrachiatum cellulase enzyme, and/or the alkaline protease is Bacillus licheniformis alkaline protease.

In some instances, a third aspect of the disclosure can be described as a process according to the first or second aspect, wherein the active agents extract comprises a hydrolyzed moringa seed powder, the hydrolyzed moringa seed powder comprising one or more of proteins, peptides, amino acids, carbohydrates, polysaccharides, monosaccharides, and lipids.

In some instances, a fourth aspect of the disclosure can be described as a process according to any one of the first through third aspects, wherein at least one of the acid solutions of one or more of steps b), h) and 1) is a hydrochloric acid solution.

In some instances, a fifth aspect of the disclosure can be described as a process according to any one of the first through fourth aspects, wherein one or both of the base solutions of steps d) and f) is a sodium hydroxide solution.

In some instances, a sixth aspect of the disclosure can be described as a process for preparing an extract of active agents for use in a healthcare product, the process comprising:
a) mixing water and moringa seed powder at about 40 °C to about 60 °C to form a solution, where the solution comprises about 80 wt % of water and about 20 wt % of moringa seed powder;
b) adjusting the pH of the solution of step a) to a pH ranging from about 2.5 to about 3.5 using an acid solution;
c) adding about 3 wt % of Aspergillus niger fungal acid protease, based on the weight of the moringa seed powder, to the product of step b) and hydrolyzing it for about 2 to about 4 hours at about 40 °C to about 60 °C;
d) adjusting the pH of the product of step c) to a pH ranging from about 3.0 to about 6.0 using a base solution;
e) adding about 2 wt % of Trichoderma longibrachiatum cellulase enzyme, based on weight of the moringa seed powder, to the product of step d) and hydrolyzing it for about 1 to about 2 hours at about 60 °C to about 67 °C;
f) adjusting the pH of the product of step e) to about 8.0 to about 10 using a base solution at about 55 °C to about 65 °C;
g) adding about 5.5 wt % of Bacillus licheniformis alkaline protease, based on weight of the moringa seed powder, to the product of step f) and hydrolyzing it for about 2 to about 6 hours at about 55 °C to about 65 °C;
h) adjusting the pH of the product of step g) to a pH<7.0 using an acid solution;
i) heating the product of step h) to about 80 °C to about 90 °C for about 15 to about 20 minutes;
j) cooling the product of step i) to a temperature of <50 °C and adjusting the pH thereof to a pH ranging from about 4.0 to about 5.0;
k) adding 0.5 wt % to 1.5 wt % of preservative, based on the total weight of the solution, to the product of step j);
l) adjusting the pH of product of step k) to 3.5 to 4.0 using an acid solution;
m) ageing product of step 1) for 0 to 10 days; and
n) filtering the product of step m) to obtain an active agents extract.

In some instances, a seventh aspect of the disclosure can be described as a process according to the sixth aspect, wherein the active agents extract comprises a hydrolyzed moringa seed powder, the hydrolyzed moringa seed powder comprising one or more of proteins, peptides, amino acids, carbohydrates, polysaccharides, monosaccharides, and lipids.

In some instances, an eighth aspect of the disclosure can be described as a process according to the sixth or seventh aspect, wherein at least one of the acid solutions of one or more of steps b), h) and 1) is a hydrochloric acid solution.

In some instances, a ninth aspect of the disclosure can be described as a process according to any one of the sixth through eighth aspects, wherein one or both of the base solutions of steps d) and f) is a sodium hydroxide solution.

In some instances, a tenth aspect of the disclosure can be described as an extract produced by a process according to any one of the first through ninth aspects.

In some instances, an eleventh aspect of the disclosure can be described as a healthcare formulation comprising an extract according to the tenth aspect.

In some instances, a twelfth aspect of the disclosure can be described as a healthcare formulation according to the eleventh aspect, wherein the healthcare formulation is a hair care formulation.

In some instances, a thirteenth aspect of the disclosure can be described as a healthcare formulation according to the eleventh or twelfth aspect, wherein the formulation further comprises one or more pharmaceutically and/or cosmetically suitable excipients.

In some instances, a fourteenth aspect of the disclosure can be described as a healthcare formulation according to any one of the eleventh through thirteenth aspects, wherein the extract is present in an amount ranging from about 0.1% to about 99% of the formulation.

In some instances, a fifteenth aspect of the disclosure can be described as a healthcare formulation according to any one of the eleventh through fourteenth aspects, wherein the formulation is in the form of a suspension, an emulsion, a gel, a spray, a bar, a stick, a balm, a cream, a lotion, a serum, or a solution.

In some instances, a sixteenth aspect of the disclosure can be described as the use of a healthcare formulation according to any one of the eleventh through fifteenth aspects for the treatment of damaged hair.

In some instances, a seventeenth aspect of the disclosure can be described as the use of a healthcare formulation according to the sixteenth aspect, wherein the treatment of damaged hair comprises at least one of
increasing an amount of disulfide groups in the damaged hair;
reducing an amount of cysteic acid residues in the damaged hair;
improving the stability of proteins of the damaged hair;
improving the mechanical strength of the damaged hair to inhibit breakage of the damaged hair.

In some instances, an eighteenth aspect of the disclosure can be described as the use of a healthcare formulation according to the sixteenth of seventeenth aspect, wherein the damaged hair was subjected to a hair coloring agent or a bleaching agent prior to use of the healthcare formulation on the damaged hair.

In some instances, a nineteenth aspect of the disclosure can be described as the use of a healthcare formulation according to the sixteenth of seventeenth aspect, wherein the damaged hair was subjected to a hair coloring agent or a bleaching agent during use of the healthcare formulation on the damaged hair.

In some instances, a twentieth aspect of the disclosure can be described as healthcare formulation comprising an extract, where the extract comprises a hydrolyzed moringa seed powder.

In some instances, a twenty-first aspect of the disclosure can be described as healthcare formulation according to the twentieth aspect, wherein the hydrolyzed moringa seed powder comprises one or more of proteins, peptides, amino acids, carbohydrates, polysaccharides, monosaccharides, and lipids.

In some instances, a twenty-second aspect of the disclosure can be described as healthcare formulation according to the twentieth or twenty-first aspect, wherein the healthcare formulation is a hair care formulation.

In some instances, a twenty-third aspect of the disclosure can be described as a healthcare formulation according to any one of the twentieth through twenty-second aspects, wherein the formulation further comprises one or more pharmaceutically and/or cosmetically suitable excipients.

In some instances, a twenty-fourth aspect of the disclosure can be described as a healthcare formulation according to any one of the twentieth through twenty-third aspects, wherein the extract is present in an amount ranging from about 0.1% to about 99% of the formulation.

In some instances, a twenty-fifth aspect of the disclosure can be described as a healthcare formulation according to any one of the twentieth through twenty-fourth aspects, wherein the formulation is in the form of a suspension, an emulsion, a gel, a spray, a bar, a stick, a balm, a cream, a lotion, a serum, or a solution.

In some instances, a twenty-sixth aspect of the disclosure can be described as the use of a healthcare formulation according to any one of the twentieth through twenty-fifth aspects for the treatment of damaged hair.

In some instances, a twenty-seventh aspect of the disclosure can be described as the use of a healthcare formulation according to the twenty-sixth aspect, wherein the treatment of damaged hair comprises at least one of
increasing an amount of disulfide groups in the damaged hair;
reducing an amount of cysteic acid residues in the damaged hair;
improving the stability of proteins of the damaged hair;
improving the mechanical strength of the damaged hair to inhibit breakage of the damaged hair.

In some instances, a twenty-eighth aspect of the disclosure can be described as the use of a healthcare formulation according to the twenty-sixth of twenty-seventh aspect, wherein the damaged hair was subjected to a hair coloring agent or a bleaching agent prior to use of the healthcare formulation on the damaged hair.

In some instances, a twenty-ninth aspect of the disclosure can be described as the use of a healthcare formulation according to the twenty-sixth of twenty-seventh aspect, wherein the damaged hair was subjected to a hair coloring agent or a bleaching agent during use of the healthcare formulation on the damaged hair.

In some instances, a thirtieth aspect of the disclosure can be described as a method for treating damaged hair, the method comprising applying a healthcare formulation according to any one of the eleventh through fifteenth and/or the twentieth through twenty-fifth aspects on damaged hair.

In some instances, a thirty-first aspect of the disclosure can be described as a method according to the thirtieth aspect, wherein the extract of the healthcare formulation penetrates into the cortex of the damaged hair during the method.

In some instances, a thirty-second aspect of the disclosure can be described as a method according to the thirtieth or thirty-first aspect, wherein the treatment of damaged hair comprises at least one of
increasing an amount of disulfide groups in the damaged hair;
reducing an amount of cysteic acid residues in the damaged hair;
improving the stability of proteins of the damaged hair; and/or
improving the mechanical strength of the damaged hair to inhibit breakage of the damaged hair.

In some instances, a thirty-third aspect of the disclosure can be described as the use of a healthcare formulation according to any one of the thirtieth through the thirty-second aspects, wherein the treatment of damaged hair comprises two or more of
increasing an amount of disulfide groups in the damaged hair;
reducing an amount of cysteic acid residues in the damaged hair;
improving the stability of proteins of the damaged hair; and
improving the mechanical strength of the damaged hair to inhibit breakage of the damaged hair.

In some instances, a thirty-fourth aspect of the disclosure can be described as the use of a healthcare formulation according to any one of the thirtieth through the thirty-third aspects, wherein the formulation is applied onto the damaged hair in more than one application step.

In some instances, a thirty-fifth aspect of the disclosure can be described as the use of a healthcare formulation according to any one of the thirtieth of thirty-fourth aspects, wherein prior to treating the damaged hair with the healthcare formulation, the hair is treated with a hair coloring agent or a bleaching agent.

In some instances, a thirty-sixth aspect of the disclosure can be described as the use of a healthcare formulation according to any one of the thirtieth of thirty-fourth aspects, wherein during treating the damaged hair with the healthcare formulation, the hair is treated with a hair coloring agent or a bleaching agent.

### BRIEF DESCRIPTION OF THE DRAWINGS

In order that the present disclosure may be readily understood, various aspects of the invention are illustrated by way of examples in the accompanying drawings, in which like parts are referred to with like reference numerals throughout.

FIG. 1 is a graphical display showing the percentage change in fluorescence intensity after one and five applications of a) a shampoo containing an active agents extract according to the present disclosure and b) a placebo shampoo, compared to an untreated initial hair sample.

FIG. 2 provides fluorescence microscopy images of cross-sectional hair fibers after one and five applications of a) a shampoo containing an active agents extract according to the present disclosure and b) a placebo shampoo, compared to an untreated initial hair sample.

FIG. 3A is a graphical display comparing the reduction in cysteic acid signal at 10⁴⁰ cm⁻¹ (asymmetric stretching of S═O bond) after one application of a) a shampoo containing an active agents extract according to the present disclosure and b) a placebo shampoo.

FIG. 3B is a graphical display comparing the reduction in disulfide signal at 525 cm⁻¹ (characteristic disulfide bond band) after one application of a) a shampoo containing an active agents extract according to the present disclosure and b) a placebo shampoo.

FIG. 4 is a graphical display comparing the percent change in total work on hair fibers after five applications of a) a shampoo containing an active agents extract according to the present disclosure and b) a placebo shampoo.

FIG. 5 is a graphical display comparing the percent change in work done after 25% strain on hair fibers after five applications of a) a shampoo containing an active agents extract according to the present disclosure and b) a placebo shampoo, compared to an untreated initial hair sample.

FIG. 6 is a graphical display comparing the thermal protection of alpha keratin and amorphous keratin resulting from treatment of hair with a) a shampoo containing an active agents extract according to the present disclosure and b) a placebo shampoo, compared to an untreated initial hair sample.

FIG. 7 provides images comparing hair fallout (breakage reduction) after treatment of a hair sample with a) a placebo shampoo, b) a commercial benchmark shampoo, and c) a shampoo containing an active agents extract according to the present disclosure.

FIG. 8 is a graphical display comparing hair fallout (breakage reduction) after treatment of a hair sample with a) a placebo shampoo, b) a commercial benchmark shampoo, and c) a shampoo containing an active agents extract according to the present disclosure.

FIG. 9 provides images comparing Scanning Electron Microscopy (SEM) images of a) an untreated hair sample, b) a hair sample treated with five applications of a placebo shampoo, c) a hair sample treated with five applications of a shampoo containing an active agents extract according to the present disclosure.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As used throughout, ranges are used as shorthand for describing each and every value that is within the range. Any value within the range can be selected as the terminus of the range. Unless otherwise specified, all percentages and amounts expressed herein and elsewhere in the specification should be understood to refer to percentages by weight.

For the purposes of this specification and appended claims, unless otherwise indicated, all numbers expressing quantities, percentages or proportions, and other numerical values used in the specification and claims, are to be understood as being modified in all instances by the term “about.” The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent, alternatively ±5 percent, alternatively ±1 percent, alternatively ±0.5 percent, and alternatively ±0.1 percent of the given numeric value provided such a deviation does not alter the end function or result of the value. Accordingly, unless indicated to the contrary, the numerical parameters set forth in this specification and attached claims are approximations that can vary depending upon the desired properties sought to be obtained by the present invention.

It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. For example, as used in this specification and the following claims, the terms “comprise” (as well as forms, derivatives, or variations thereof, such as “comprising” and “comprises”), “include” (as well as forms, derivatives, or variations thereof, such as “including” and “includes”) and “has” (as well as forms, derivatives, or variations thereof, such as “having” and “have”) are inclusive (i.e., open-ended) and do not exclude additional elements or steps. Accordingly, these terms are intended to not only cover the recited element(s) or step(s), but may also include other elements or steps not expressly recited. Furthermore, as used herein, the use of the terms “a” or “an” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “moringa seed powder” means a powder recovered after the extraction of oils from seeds of Moringa oleifera. The moringa seed powder is therefore enriched with non-oil components of moringa seed.

Various aspects of the disclosure are directed to processes of extracting of active agents and using the active agents extracts as is or in hair care formulations for treating oxidatively damaged hair. On application to damaged hair, active agents extracts according to the disclosure have been found to reverse the oxidative damage such as in hair proteins or in skin proteins and provide protein repair at molecular level, leading to healthier hair with increased tensile strength, less breakage, improved appearance, and smooth feel without leaving the hair feeling greasy. Active agents extracts according to the disclosure have also been found to provide hair with thermal protection and long lasting moisturization. Relatedly, it has been found that hair care formulations, comprising active agents extracts according to various aspects of the disclosure, contain one or more compounds that penetrate into hair cortex, induce reversal of the cysteine oxidative damages, namely cysteic acid residues, promote disulfide bond formation, improve the protein stability, strengthens the hair fiber, and protect the hair from further oxidative damage.

Active agents extracts described herein can be used as-is or incorporated or formulated into a various healthcare composition or formulations accordingly. For example, active agents extracts described herein can be used as-is or as a component of multicomponent compositions or formulations for use in various healthcare applications such as hair care, skin care, body care applications. Compositions and formulations comprising active agents extracts described herein can take various forms including, but not limited to, suspensions, emulsions, gels, sprays, bars, sticks, balms, creams, lotions, serums and solutions.

An exemplary process for the extraction of active agents, according to various aspect of the disclosure, comprises:
a) preparing a suspension by mixing a solution of water and moringa seed powder (about 75 wt % to about 87 wt % of water and about 13 wt % to about 25 wt % moringa seed powder, based on the total weight of the solution) at about 40 °C to about 60 °C;
b) adjusting the pH of suspension of step a) to a pH of about 2.5 to about 3.5 using an acid solution;
c) adding about 1.5 wt % to about 4 wt % Aspergillus niger fungal acid protease, based on the weight of the moringa seed powder, to the pH-adjusted suspension of step b) to form a mixture, and hydrolyzing the mixture for about 2 to about 4 hours at about 40 °C to about 60 °C;
d) adjusting the pH of the hydrolyzed mixture of step c) to a pH of about 3.0 to about 6.0 using a base solution;
e) adding about 0.5 wt % to about 3 wt % Trichoderma longibrachiatum cellulase enzyme, based on the weight of moringa seed powder, to the pH-adjusted hydrolyzed mixture of step d) and hydrolyzing it for about 1 to about 2 hours at about 60 °C to about 67 °C;
f) adjusting the pH of the hydrolyzed mixture of step e) to a pH of about 8.0 to about 10 using a base solution at about 55 °C to about 65 °C;
g) adding about 3.5 wt % to about 6.5 wt % Bacillus licheniformis alkaline protease, based on weight of moringa seed powder to pH-adjusted mixture of step f) and hydrolyzing it for about 2 to about 6 hours at about 55 °C to about 65 °C;
h) adjusting the pH of hydrolyzed mixture of step g) to <7.0 using an acid solution;
i) heating the pH-adjusted mixture of step h) to about 80 °C to about 90 °C for about 15 to about 20 minutes;
j) cooling the heated mixture of step i) to <50 °C and adjusting the pH of mixture to a pH between about 4.0 to about 5.0.
k) adding about 0.5 wt % to about 2.0 wt % of a preservative, based on the total weight of the cooled mixture of step j), to the cooled mixture of step j),
l) adjusting the pH of the mixture of step k) to about 3.5 to about 4.0 using an acid solution, m) ageing the pH-adjusted mixture of step 1) for a period of time ranging from 0 to about 50 days, and
n) filtering the aged mixture of step m) to obtain an active agents extract.

In some instances, the suspension of step a) can be formed to have from about 76 wt % to about 85 wt % of water and from about 15 wt % to about 24 wt % moringa seed powder, alternatively from about 77 wt % to about 83 wt % of water and from about 17 wt % to about 23 wt % moringa seed powder, alternatively from about 78 wt % to about 82 wt % of water and from about 18 wt % to about 22 wt % moringa seed powder, alternatively from about 79 wt % to about 81 wt % of water and from about 19 wt % to about 20 wt % moringa seed powder, and alternatively about 80 wt % of water and 20 wt % of moringa seed powder.

In some instances, the Aspergillus niger fungal acid protease is added, in step c), to the pH-adjusted suspension of step b) in an amount ranging from about 1.5 wt % to about 4.0 wt %, alternatively from about 2.0 wt % to about 4.0 wt %, alternatively from about 2.25 wt % to about 3.75 wt %, alternatively from about 2.5 wt % to about 3.5 wt %, alternatively from about 2.75 wt % to about 3.25 wt %, and alternatively about 3.0 wt %, based on the weight of the moringa seed powder used in step a). In some instances, fungal acid proteases other than Aspergillus niger fungal acid protease may be used. In some instances, an aspartic protease may be used in step c) instead of or in combination with a fungal acid protease. Examples of suitable aspartic proteases include, but are not limited to, pepsin, chymosin, renin, cathepsins, and HIV protease.

In some instances, the Trichoderma longibrachiatum cellulase enzyme is added, in step e), to the pH-adjusted hydrolyzed mixture of step d) in an amount ranging from about 0.5 wt % to about 3.0 wt %, alternatively from about 1 wt % to about 2.5 wt %, alternatively from about 1.5 wt % to about 2.5 wt %, alternatively from about 1.75 wt % to about 2.25 wt %, and alternatively about 2.0 wt %, based on the weight of moringa seed powder used in step a). In some instances, a cellulase enzyme other than Trichoderma longibrachiatum cellulase enzyme may be used. In some instances, beta-glucanase, cellulose phosphorylase, or cellobiose dehydrogenase may be used in step d) instead of or in combination with Trichoderma longibrachiatum cellulase enzyme.

In some instances, the Bacillus licheniformis alkaline protease is added, in step g), to the pH-adjusted mixture of step f) in an amount ranging from about 4.0 wt % to about 6.5 wt %, alternatively from about 4.5 wt % to about 6.25 wt %, alternatively from about 5.0 wt % to about 6.0 wt %, alternatively from about 5.25 wt % to about 5.75 wt %, and alternatively about 5.5 wt %, based on the weight of moringa seed powder used in step a). In some instances, an alkaline protease other than Bacillus licheniformis alkaline protease may be used. In some instances, alkaline proteases in the ArpE family of enzymes (such as, for example, subtilisin, Alcalase, Esperase, and Savinase) may be used in step f) instead of or in combination with Bacillus licheniformis alkaline protease.

In some instances, the acid of the acid solutions of steps b), h) and 1) is the same. In some instances, the acid of the acid solutions of steps b), h) and 1) is different. In some instances, the acid of the acid solutions of one or more of steps b), h) and 1) is hydrochloric acid.

In some instances, the base of the base solutions of steps d) and f) is the same. In some instances, the base of the base solutions of steps d) and f) is different. In some instances, the base of the base solutions of one or both of steps d) and f) is sodium hydroxide.

In some instances, the pH adjustment of step j) is performed using an acid solution, which may be the same or different than the acid solution(s) used in one or more of steps b), h) and 1). In some instances, the pH adjustment of step j) is performed using a base solution, which may the same of different than the base solution(s) use in one or both of steps d) and f).

In some instances, the ageing of step m) is performed for a period of time ranging from 0 to about 40 days, alternatively from 0 to about 30 days, alternatively from 0 to about 20 days, and alternatively from 0 to about 10 days. In some instances, the ageing of step m) is performed for a period of time of at least 1 day. In some instances, the ageing of step m) is performed for a period of time of at least 2 days, alternatively at least 3 days, alternatively at least 4 days, alternatively at least 5 days and alternatively at least 10 days.

The type of preservative added in step k) is not particularly limited. In some instances, the use of preservatives such as sodium benzoate potassium sorbate, phenoxyethanol, caprylyl glycol, ethylhexyl glycerin, or combinations of the foregoing, is preferred. Other forms of preservatives can also be used for anti-microbial effectiveness.

Active agents extracts produced according to various aspects of the disclosure have been found stable and effective for a period of at least 36 months at 20 °C to 30 °C and pH range of 4.0-7.0. Active agents extracts produced according to various aspects of the disclosure are based on hydrolyzed moringa seed powder and comprise one or more of proteins, peptides, amino acids, carbohydrates, polysaccharides, monosaccharides, and lipids. Active agents extracts produced according to various aspects of the disclosure have been found to contain enzymatically hydrolyzed carbohydrates, enzymatically hydrolyzed proteins, and other bioactive components of moringa oleifera seed. In some instances, active agents extracts produced according to various aspects of the disclosure are suspended in an aqueous solution and preserved with 1%-2% sodium benzoate.

Active agents extracts produced according to various aspects of the disclosure have been found to exhibit a molecular weight distribution ranging from around 100 Da to around 3,500 Da.

Active agents extracts prepared according to various aspects of the disclosure can be used as-is or incorporated or formulated into various healthcare compositions or formulations accordingly. For example, active agents extracts described herein can be used as-is or as a component of multicomponent compositions or formulations for use in various healthcare applications such as hair care, skin care, and body care applications. Compositions and formulations comprising active agents extracts described herein can take various forms including, but not limited to, suspensions, emulsions, gels, sprays, bars, sticks, balms, creams, lotions, serums and solutions. Active agents extracts prepared according to various aspects of the disclosure can be used can in compositions or formulations at various concentrations ranging from about 0.1% (w/w) to 100% (w/w).

Active agents extracts prepared according to various aspects of the disclosure can be combined with one or more pharmaceutically and/or cosmetically acceptable carriers and/or excipients that are considered safe and effective to human hair, skin, and/or human scalp, and may be administered to an individual's hair without causing undesirable side effects, such as burning, itching, and/or redness, or similar adverse reactions. These excipients may contain water phase, viscosity controlling ingredients such as carbomers, gums, and polymers; oil phase ingredients such as triglycerides and short chain fatty acids; emulsifiers such as long chain fatty alcohols, fatty acids, multiply substituted and amphiphilic glycerides; pH adjusters such as inorganic hydroxides, inorganic acids and organic acids; metal chelators such as EDTA, gluconates, phytates and citrates; preservatives such as glycols, organic alcohols, and organic acids.

### EXAMPLES

General Experimental Parameters. Double bleached Caucasian hair swatches were prewashed with 10% sodium laureth sulfate (SLES)/water for 1 minute, followed by 30 seconds of rinse under free-flowing water. The hair swatches were towel dried and equilibrated in a standardized environment at 55±5% relative humidity and 22±2 °C for 24 hours prior to the study. The hair swatches were then washed with Placebo Shampoo vs 1% (w/w) Active Agents Shampoo for 1 minute, followed by 30 seconds of rinse under free-flowing water. The hair swatches were then towel dried and incubated in a standardized environment at 55±5% relative humidity and 22±2 °C for 24 hours. The application samples underwent five cycles of shampoo, dry, and incubate 24 hours process. The resulting hair swatches were used for further analyses.

As referred to in the Examples below, all Active Agents formulations (extracts, shampoos, conditioners) are according to the invention.

### Example 1

In this example, a process of extraction of hair care active agents is presented. The process included the following steps:
a) forming a suspension by mixing 80 wt % of water and 20 wt % of moringa seed powder, based on the total weight of a suspension, at 40 °C;
b) adjusting the pH of the suspension of step a) to 2.5 using an acid solution;
c) adding 3 wt % Aspergillus niger fungal acid protease, based on the weight of the moringa seed powder, to the product of step b) and hydrolyzing it for 4 hours at 40 °C;
d) adjusting the pH of the product of step c) to 3.0 using a base solution;
e) adding 2 wt % Trichoderma longibrachiatum cellulase enzyme, based on the weight of the moringa seed powder, to the product of step d) and hydrolyzing it for 2 hours at 60 °C;
f) adjusting the pH of the product of step e) to 8.0 using a base solution at 55 °C;
g) adding 5.5 wt % Bacillus licheniformis alkaline protease, based on weight of moringa seed powder, to the product of step f) and hydrolyzing it for 6 hours at 55 °C;
h) adjusting the pH of the product of step g) to <7.0 using an acid solution;
i) heating the product of step h) to 80 °C to 90 °C for 15 to 20 minutes;
j) cooling the product of step i) to <50 °C and adjusting the pH of solution at 4.0 to 5.0;
k) adding 0.5 wt % to 1.5 wt % of preservative to the product of step j), based on the total weight of the product of step j);
l) adjusting the pH of product of step k) to 3.5 to 4.0 using an acid solution;
m) ageing the product of step 1) for 0 to 10 days; and
n) filtering the product of step m) to obtain an active hair agents extract.

The hair care active agents extract produced according to this example is formulated into hair care compositions and further evaluated in the subsequent Examples. As evidenced by the subsequent Examples, compositions according to the invention (which include the active agents extract produced in this Example, upon on application to damaged hair, re-bonds the damaged hair, reverses cysteine oxidative damage, and repairs disulfide bonds of cysteine present in the hair.

### Example 2—Hair Cortex Penetration of Active Agents Shampoo vs Placebo Shampoo Methods

The swatches treated with Placebo Shampoo vs 1% (w/w) Active Agents Shampoo were cross-sectionally cut using a microtome, dyed with Rhodamine B, and evaluated under fluorescence microscopy.

### Results

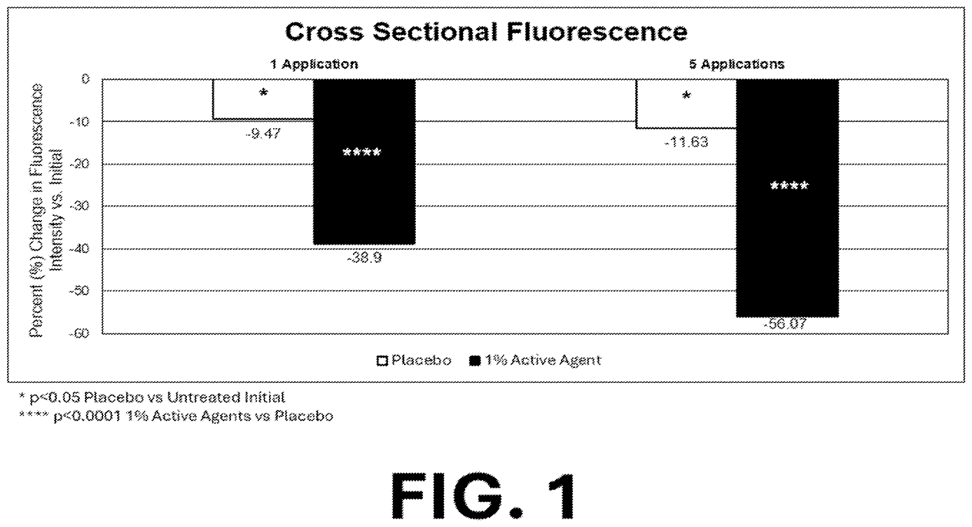

The substantivity and penetration of Active Agents on bleached Caucasian hair swatches versus placebo treatments compared to initial hair samples are illustrated in FIG. 1. Fluorescence intensity was decreased from the untreated initial hair swatches by −9.47% for placebo shampoo treated hair (*p<0.05 Placebo treated vs Untreated Initial hair samples) and −38.9% for Active Agents shampoo treated hair (****p<0.0001 1% Active Agents treated vs Untreated Initial hair samples and Placebo treated hair samples), after just one application. Decrease in fluorescence intensity after five applications was-11.63% for placebo shampoo treated hair (*p<0.05 Placebo treated vs Untreated Initial hair samples) and −56.07% (****p<0.0001 1% Active Agents treated hair samples vs Untreated Initial and Placebo treated hair samples) for Active Agents Shampoo treated hair. The degree to which Active Agents decrease the fluorescence in both one and five applications indicate penetration into the hair fibers, resulting in effective entry into the cortex.

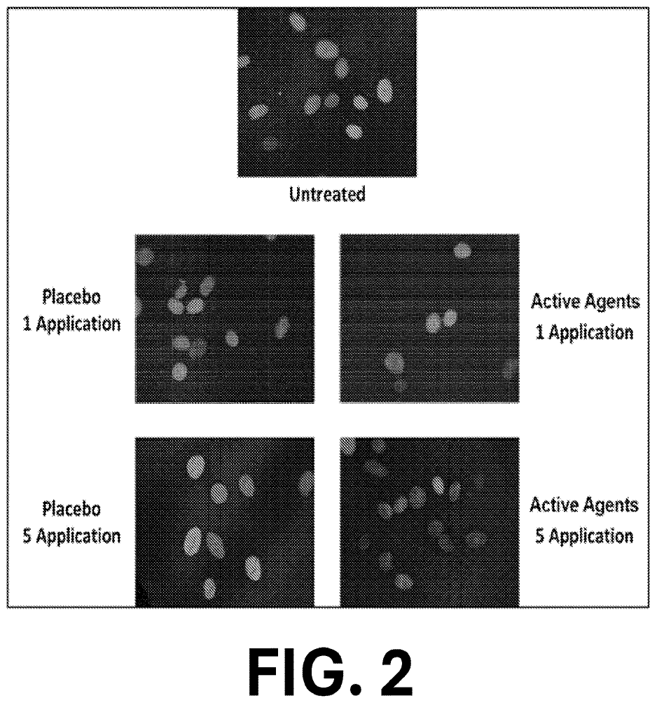

FIG. 2 provides fluorescence microscopy images of cross-sectional hair fibers after one and five applications of the 1% (w/w) Active agents shampoo and placebo shampoo treatments compared to untreated initial hair fibers. Results show that Active Agents have strong affinity to the hair fibers, leaving fewer sites for the Rhodamine B dye to bind. The uniform reduction in the fluorescence intensity indicates that the Active Agents can penetrate into various layers of the hair fiber or cortex.

### Example 3—Hair Cysteic Acid Repair Disulfide Formation by Active Agents Shampoo vs Placebo Shampoo

### Methods

The swatches treated with Placebo Shampoo vs. 1% (w/w) Active Agents Shampoo (one application) were analyzed via Fourier-transform Infrared Spectroscopy-Attenuated Total Reflectance (FTIR-ATR) using an FTIR (Perkin Elmer) with an ATR cell (Pike Technologies) and ZnSe crystal.

### Results

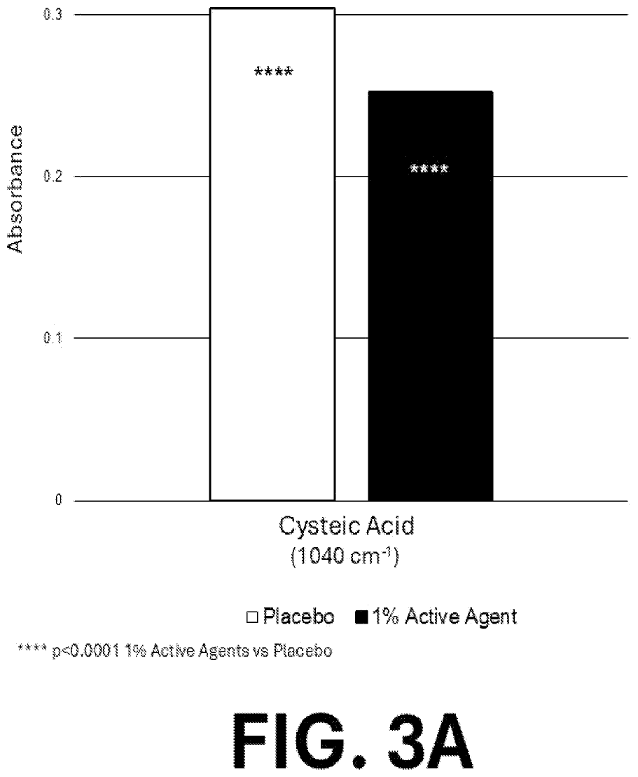

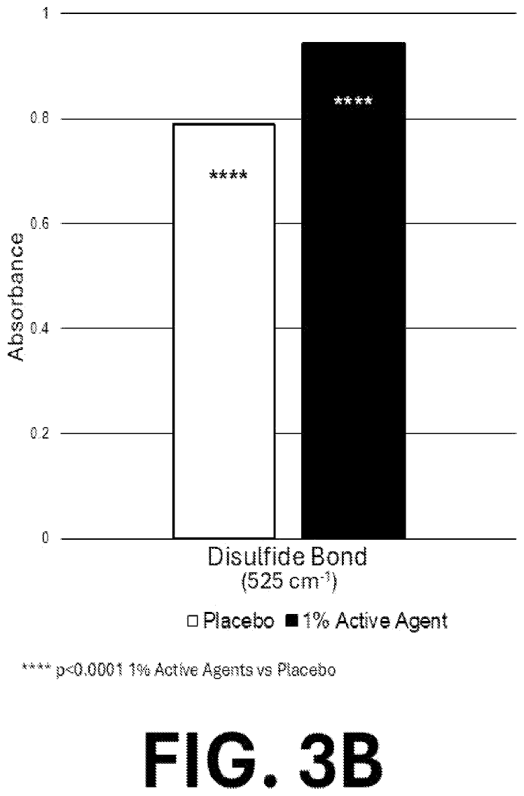

The Cysteic acid repair and disulfide bond restoration according to ATR-FTIR study are illustrated in FIGS. 3A and 3B, respectively. As shown in FIG. 3A, the cysteic acid content monitored at 10⁴⁰ cm⁻¹ (asymmetric stretching of S═O bond) shows a 17% reduction by Active Agents Shampoo treatment vs Placebo Shampoo after one application, with statistical significance of ****p<0.0001. The disulfide content monitored at 525 cm⁻¹ (characteristic disulfide bond band) shows a 20% increase by Active Agents Shampoo treatment vs Placebo Shampoo, after one application statistical significance of ****p<0.0001.

### Example 4—Hair Fiber Tensile Strength Improvement by Active Agents Shampoo vs Placebo Shampoo

### Methods

The swatches treated with 1% (w/w) Active Agents Shampoo (five applications) vs Placebo Shampoo were analyzed using Dia-Stron MTT175 Miniature Tensile Tester & Dia-Stron Crimp Assembly System.

### Results

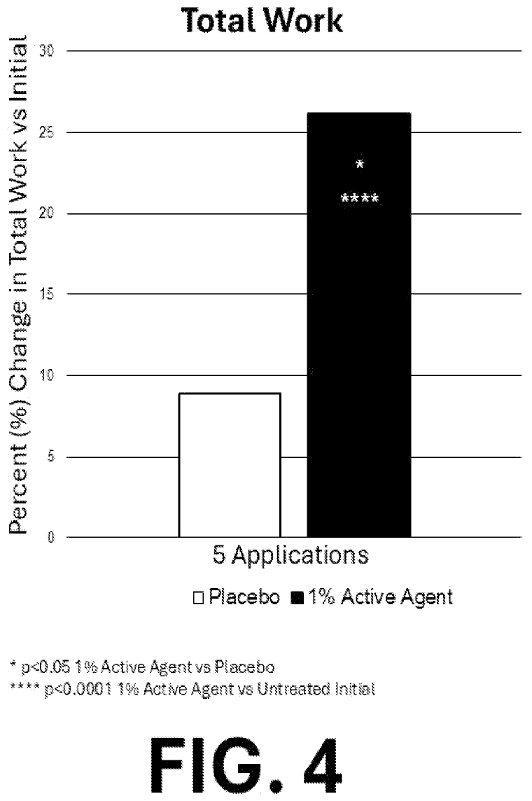

Tensile strength testing for total work to reach the break point was reported as percent change from initial readings to five applications of each respective treatment, as shown in FIG. 4. Placebo treatment resulted in an 8.87% change from the baseline, with no statistical significance. Active Agents Shampoo Treatment effectively increased the total work required by 26.22% (*p<0.05 Active Agents vs Placebo and ****p<0.0001 Active Agents vs Initial) with prominent statistical significance. Through this study, we see Active Agents performance in increasing strength of hair fibers through the significant increase in force required to reach the breaking point.

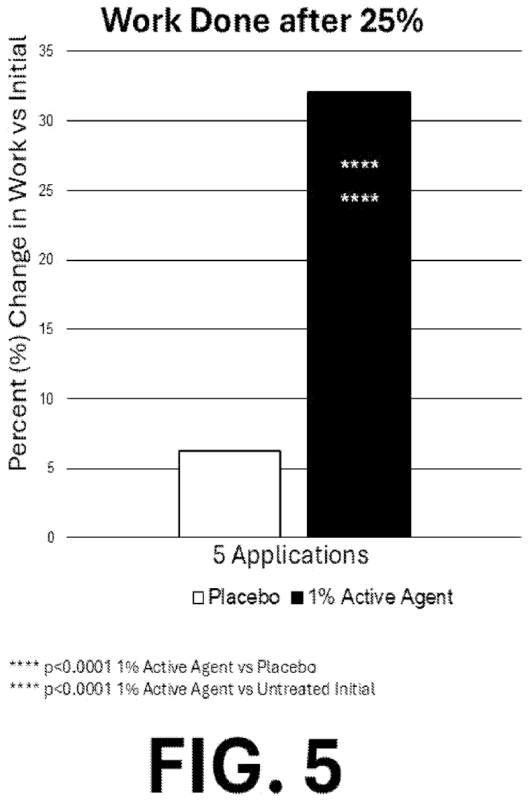

The “yield” region, after strain of 25% was assessed after five applications of Active Agents Shampoo Treatment vs Placebo Shampoo Treatment. The results in FIG. 5 illustrate the placebo treated hair fiber had a 6.28% change from initial, with no statistical significance. The Active Agents treated hair had a 32.08% change from initial, with a statistical significance value of ****p<0.0001 versus placebo and initial. Thus, the Active Agents treatment increased the force required within the “yield” region.

### Example 5—Hair Protein Stability Enhancement by Active Agents Shampoo Treatment vs Placebo Shampoo Treatment

### Methods

10 mg samples of each swatch treated with 1% (w/w) Active Agents Shampoo (one application) vs Placebo Shampoo were chopped and analyzed using Thermogravimetric Differential Scanning calorimetry (DSC; Hitachi High-Tech Sciences TA7000 Series Simultaneous Thermogravimetric Analyzer, STA7200).

### Results

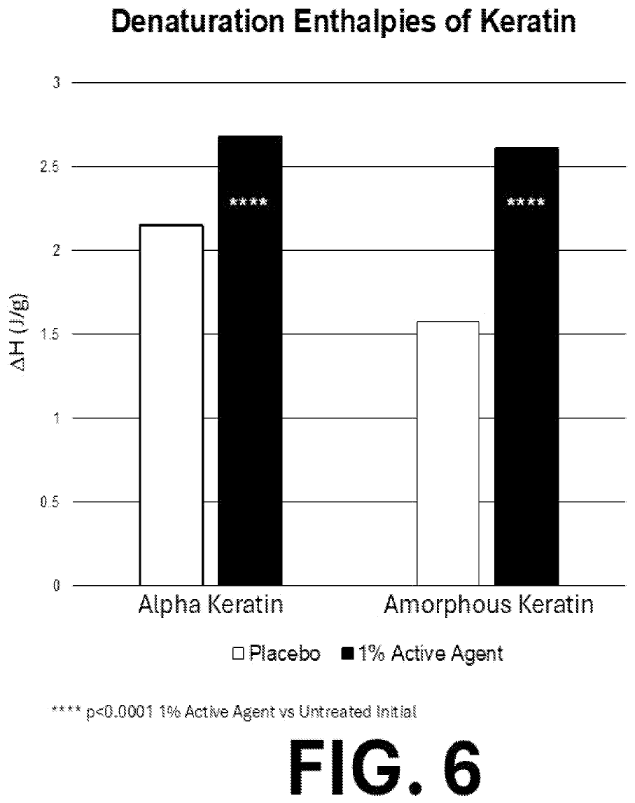

The DSC study showed that both the a-keratin and amorphous keratin ΔH_{d} (denaturation enthalpy) values were significantly higher for the swatches treated with Active Agents vs Placebo (FIG. 6). The active agents treated hair tresses showed 33% and 66% higher ΔH_{d} for a-keratin phase and amorphous keratin phase, respectively, with the statistical significance of ****p<0.0001, compared to placebo groups. The increased α-keratin ΔH_{d} value indicates that alpha structure of keratin is more strongly retained in the hair swatches treated with active agent. It also indicates that external water molecules are bound to keratin more tightly. The increased amorphous keratin ΔH_{d} value indicates that more energy is required to completely denature the keratins in the hair swatches treated with the active agent. The internal hydrogen bonds of keratin are more strongly retained thus keratin is more resilient to thermal denaturation. The results indicate that the active agent treatment enhances the hair protein stability, enabling the proteins to be more resistant toward thermal denaturation.

### Example 6—Hair Fallout/Breakage Reduction by Active Agents Treatment vs Placebo Treatment

### Methods

The virgin (untreated initial) hair swatches are treated with a commercial hair bleach kit following the instruction on the commercial hair bleach kit label. The hair bleach treatment time was increased to 24 hours, instead of 20-40 minutes, to subject the hair swatches to extreme harsh conditions. 7 wt % of water was added to the hair bleach kit to generate the placebo formulation sample. A neat solution of 7 wt % of salon professional grade hair bond builder product was added to the hair bleach kit to generate the commercial benchmark formulation sample. A neat solution of 7 wt % of active agents extract was added to the hair bleach kit to generate an inventive Active Agents-containing formulation sample according to various aspects of the disclosure.

After 24 hours of hair bleaching treatment using the placebo, the commercial benchmark, and the Active Agents-containing formulation on three separate hair swatches, the hair swatches were rinsed with running water for 1 minute, followed by towel drying. The hair swatches were then wet-combed 10 times. The hair fallouts/breakages resulting from the wet-combing were then collected and dried. The hair swatches were then equilibrated and dried in a humidity chamber (55 ±5% relative humidity and 22±2 °C) for 24 hours. The hair swatches were then dry-combed 10 times. The hair fallouts/breakages resulting from the dry-combing were then collected and combined with the collected and dried hair fallouts/breakage samples produced from the wet-combing step. The combined/total hair fallout/breakage weight was then measured using an analytical balance.

### Results

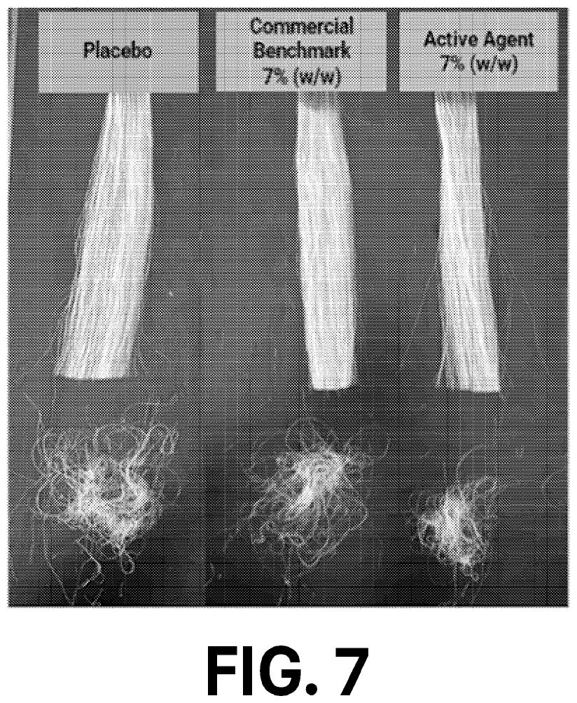

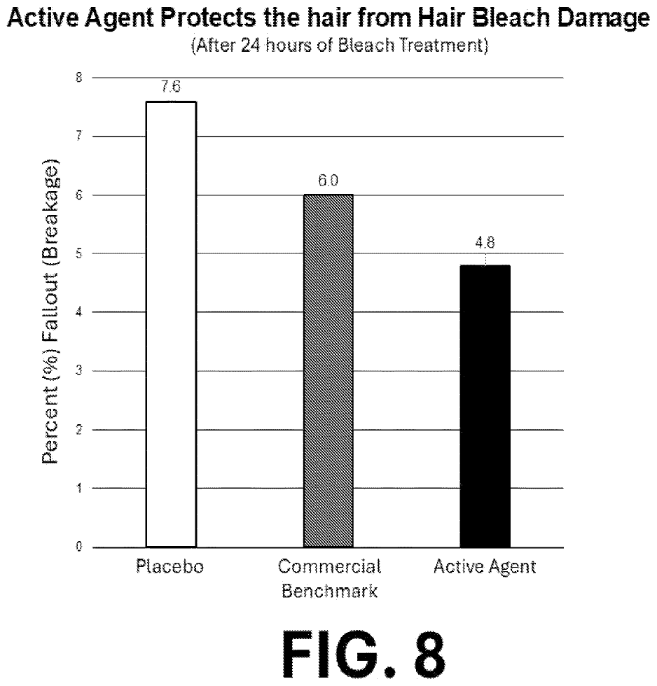

FIG. 7 is an image providing a visual comparison of the hair samples treated with, from left to right in the image, the placebo formulation sample, the commercial benchmark formulation sample and the Active Agents-containing formulation sample, respectively. FIG. 8 is a graphical display comparing the degree of hair fallout (breakage reduction) of the hair samples treated with, from left to right in the image, the placebo formulation sample, the commercial benchmark formulation sample and the Active Agents-containing formulation sample, respectively. As is clear in the qualitative and quantitative data provided in FIGS. 7 and 8, the hair swatch treated with the Active Agents-containing formulation showed the least amount of hair fallout/breakage (4.8 wt %) compared to the hair samples treated with the placebo formulation sample (7.6 wt %) and the commercial benchmark formulation sample (6.0 wt %). The results demonstrate the ability of the Active Agents-containing formulations according to various aspects of the disclosure to protect the hair from fallout/breakage caused by 24 hours of commercial hair bleach kit exposure.

### Example 7—Hair Fiber Surface Enhancement by Active Agents Shampoo Treatment vs Placebo Shampoo Treatment

### Methods

The hair fiber samples were obtained from each hair swatch treated with 1% (w/w) Active Agents Shampoo (one application) vs Placebo Shampoo. The treated hair fiber samples were mounted on PELCO Tabs™ Carbon Conductive Tabs, fixed on aluminum mount. The hair fiber samples were analyzed using NanoScience Phenom Pure Scanning Electron Microscopy.

### Results

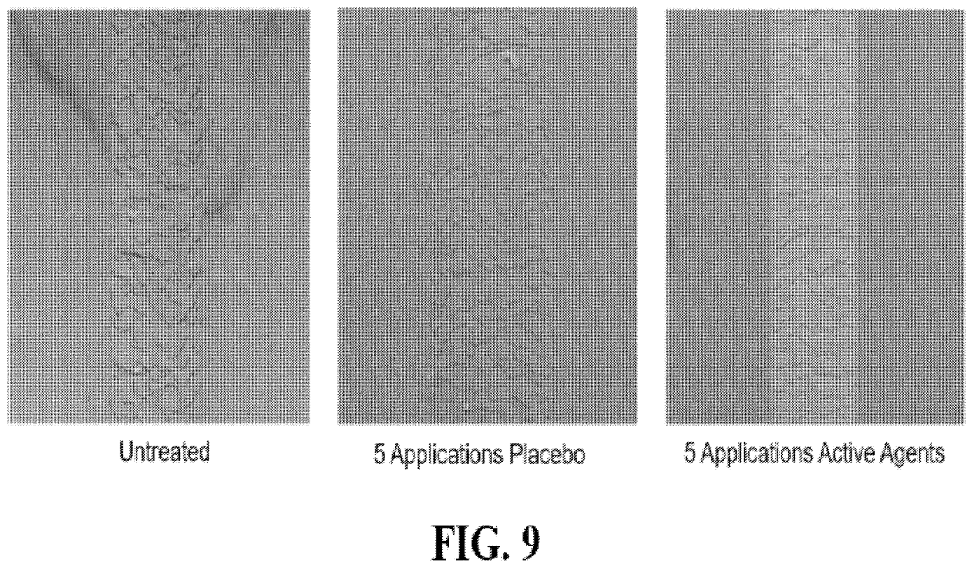

The scanning electron microscope (SEM) images revealed the baseline, double bleached hair exhibited severe damage, characterized by lifted cuticles and an overall compromised appearance (FIG. 9; left-hand image). After five applications of the placebo shampoo, there was minimal improvement observed (FIG. 9; middle image). The structural integrity of the hair fibers remained largely unchanged. Conversely, after five applications of the 1% Active Agents Shampoo formulation, a notable transformation was evident (FIG. 9; right-hand image). The cuticle layer appeared even, and the overall appearance of the hair indicated signs of substantial recovery, indicating the effectiveness of the protein-based treatment in restoring hair health.

### Example 8—Hair Shampoo Composition Containing Active Agents

In this Example, an exemplary inventive hair shampoo composition according to various aspects of the disclosure is provided.

| Ingredients | % w/w |
|---|---|
| Water | QS |
| Glycerin | 3.00 |
| Cocamidopropyl Betaine | 10.00 |
| Sodium Laureth Sulfate | 35.00 |
| Preservative Blend (Phenoxyethanol, Benzoic | 1.00 |
| Acid, Capryloyl Glycine, Undecylenoyl | |
| Glycine) | |
| Active Agents extract | 0.50-5.00 |
| Sodium Chloride | 0.60 |

### Example 9—Leave-on Hair Conditioner Composition Containing Active Agents

In this Example, an exemplary inventive hair conditioner composition according to various aspects of the disclosure is provided.

| Ingredients | % w/w |
|---|---|
| Water | QS |
| Glycerin | 2.00 |
| Carbomer | 0.20 |
| Tetrasodium Glutamate Diacetate | 0.10 |
| Emulsifier Blend (Glyceryl Stearate and | 2.00 |
| Cetearyl Alcohol and Sodium Stearoyl | |
| Lactylate) | |
| Caprylic/Capric Triglyceride | 2.00 |
| PEG-60 Hydrogenated Castor Oil | 1.00 |
| Sodium Hydroxide 10% Solution | 0.30 |
| Preservative Blend (Benzyl Alcohol, Water, | 1.00 |
| Potassium Sorbate, Sodium Benzoate) | |
| Active Agents extract | 0.50-5.00 |

### Example 10—Hair Strengthening Serum Composition Containing Active Agents

In this Example, an exemplary inventive hair serum composition according to various aspects of the disclosure is provided.

| Ingredients | % w/w |
|---|---|
| Water | QS |
| Tetrasodium Glutamate Diacetate | 0.10 |
| Glycerin | 2.00 |
| Guar Gum | 0.75 |
| Panthenol | 0.25 |
| Behentrimonium Chloride | 1.00 |
| Cetearyl Alcohol | 1.5 |
| Hydrogenated Ethylhexyl Olivate, Hydrogenated | 2.00 |
| Olive Oil Unsaponifiables | |
| C9-12 Alkane | 2.00 |
| Panthenyl Hydroxypropyl Steardimonium Chloride | 0.50 |
| Active Agents extract | 1.00 |
| Phenoxyethanol, Caprylyl Glycol | 0.50-5.00 |
| Citric Acid | 0.01 |

### Example 11—Hair Primer Composition Containing Active Agents

In this Example, an inventive hair primer composition according to various aspects of the disclosure is provided.

| Ingredients | % w/w |
|---|---|
| Water | QS |
| Tetrasodium Glutamate Diacetate | 0.20 |
| Glycerin | 3.00 |
| Hydroxyethyl cellulose | 0.25 |
| Guar Hydroxypropyltrimonium Chloride | 0.20 |
| Panthenol | 0.10 |
| Butylene Glycol | 2.00 |
| Behentrimonium Chloride | 3.00 |
| Cetearyl alcohol | 5.00 |
| Cetyl Palmitate | 3.00 |
| Glyceryl Stearate | 2.00 |
| Hydrogenated Ethylhexyl Olivate, Hydrogenated | 1.00 |
| Olive Oil Unsaponifiables | |
| Active Agents extract | 0.50-5.00 |
| Phenoxyethanol (and) Caprylyl Glycol | 1.00 |
| Citric Acid | 0.17 |

### Example 12—Hair Lathering Shampoo Composition Containing Active Agents

In this Example, another exemplary inventive hair shampoo composition according to various aspects of the disclosure is provided.

| Ingredients | % w/w |
|---|---|
| Water | QS |
| Tetrasodium Glutamate Diacetate | 0.20 |
| Panthenol | 0.20 |
| Sodium Lauroyl Sarcosinate | 12.00 |
| Cocoamidopropyl Betaine | 14.00 |
| Decyl Glycoside | 12.00 |
| PEG-120 Methyl Glucose Dioleate | 2.30 |
| Active Agents extract | 0.50-5.00 |
| Phenoxyethanol (and) Benzoic Acid | 1.20 |
| (and) Capryloyl Glycine (and) | |
| Undecylenoyl | |
| Glycine | |
| Citric Acid | 1.00 |

### Example 13—Hair Strengthening Conditioner Composition Containing Active Agents

In this Example, another exemplary inventive hair conditioner composition according to various aspects of the disclosure is provided.

| Ingredients | % w/w |
|---|---|
| Water | QS |
| Tetrasodium Glutamate Diacetate | 0.30 |
| Panthenol | 0.10 |
| Glycerin | 2.00 |
| Hydroxyethylcellulose | 0.30 |
| Behentrimonium Chloride | 1.50 |
| Cetearyl Alcohol | 5.00 |
| Cetyl Palmitate | 1.00 |
| Glyceryl Stearate | 2.00 |
| Hydrogenated Ethylhexyl Olivate (and) | 0.10 |
| Hydrogenated Olive Oil Unsaponifiables | |
| Olea Europaea (Olive) Oil | 0.60 |
| Aloe Barbadensis Leaf Juice | 1.00 |
| Water, Citric Acid | 0.20 |
| Active Agents extract | 0.50-5.00 |
| Phenoxyethanol | 1.00 |

### Example 14—Hair Fallout/Breakage Protection Active Agent (Protection from Commercial Hair Bleaching Damage)

In this Example, an exemplary inventive hair protection composition according to various aspects of the disclosure is provided.

| Ingredients | % w/w |
|---|---|
| Active Agents extract | 100 |

1-19. (canceled)

20. A healthcare formulation comprising an extract, where the extract comprises a hydrolyzed moringa seed powder.

21. The healthcare formulation of claim 20, wherein the hydrolyzed moringa seed powder comprises one or more of proteins, peptides, amino acids, carbohydrates, polysaccharides, monosaccharides, and lipids.

22. The healthcare formulation of claim 20, wherein the healthcare formulation is a hair care formulation.

23. The healthcare formulation of claim 20, wherein the formulation further comprises one or more pharmaceutically and/or cosmetically suitable excipients.

24. The healthcare formulation of claim 20, wherein the extract is present in an amount ranging from about 0.1% to about 99% of the formulation.

25. The healthcare formulation of claim 20, wherein the formulation is in the form of a suspension, an emulsion, a gel, a spray, a bar, a stick, a balm, a cream, a lotion, a shampoo, a conditioner, a serum, or a solution.

26-29. (canceled)

30. A method for treating damaged hair, the method comprising applying a healthcare formulation according to claim 20 on damaged hair.

31. The method of claim 30, wherein the extract of the healthcare formulation penetrates into the cortex of the damaged hair during the method.

32. The method of claim 30, wherein the treatment of the damaged hair comprises at least one of
increasing an amount of disulfide groups in the damaged hair;
reducing an amount of cysteic acid residues in the damaged hair;
improving the stability of proteins of the damaged hair; and/or
improving the mechanical strength of the damaged hair to inhibit breakage of the damaged hair.

33. The method of claim 30, wherein the healthcare formulation is applied onto the damaged hair in more than one application step.

34. The method claim 30, further comprising, prior to treating the damaged hair with the healthcare formulation, treating the hair with a hair coloring agent or a bleaching agent.

35. The method claim 30, further comprising, during treating the damaged hair with the healthcare formulation, treating the hair with a hair coloring agent or a bleaching agent.

36. The healthcare formulation of claim 22, wherein the hair care formulation is a shampoo.

37. The healthcare formulation of claim 22, wherein the hair care formulation is a conditioner.

38. The healthcare formulation of claim 22, wherein the hair care formulation is a hair serum or hair primer.

39. The healthcare formulation of claim 20, wherein the healthcare formulation further comprises a water phase, an oil phase, and an emulsifier.

40. The healthcare formulation of claim 20, wherein the healthcare formulation further comprises at least one of pH adjuster, a metal chelator, and a preservative.

41. The healthcare formulation of claim 20, wherein the healthcare formulation further comprises a pH adjuster, a metal chelator, and a preservative.

42. The healthcare formulation of claim 20, wherein the healthcare formulation comprises the extract suspended in an aqueous solution.

43. The healthcare formulation of claim 20, wherein the extract exhibits a molecular distribution ranging from about 100 Da to about 3,500 Da.
